(19) United States
(12) Patent Application Publication
(10) Pub. No.: US 2026/0007606 A1
(43) Pub. Date: Jan. 8, 2026

(54) NAPROXEN SODIUM TABLETS PRODUCED USING A CONTINUOUS PROCESS

(71) Applicant: BAYER HEALTHCARE LLC, Whippany, NJ (US)

(72) Inventors: Satish V. KAMATH, Budd Lake, NJ (US); Frank EISMANN, NorthRhine-Westphalia (DE)

(21) Appl. No.: 18/875,229

(22) PCT Filed: Jun. 20, 2023
(86) PCT No.: PCT/US2023/068745

§ 371 Date: Dec. 16, 2024

Related U.S. Application Data
(60) Provisional application No. 63/353,751, filed on Jun. 20, 2022.

(51) Int. Cl. A61K 9/20; A61K 31/192

(52) U.S. Cl. CPC A61K 9/2059; A61K 9/2018; A61K 9/2095; A61K 31/192

(57) ABSTRACT

Provided are oral tablets comprising naproxen sodium, mannitol, a superdisintegrant, and a lubricant. The oral tablets described herein to not comprise a glidant (e.g. colloidal silicon dioxide). These oral tablets may be manufactured using the process of mixing naproxen sodium, mannitol, a superdisintegrant, and magnesium stearate in a continuous in-line mixer to form a tableting mixture; transferring the tableting mixture to a tablet press including three or more compression rollers; and pressing the tableting mixture into naproxen sodium tablets.

### CROSS-REFERENCE TO RELATED APPLICATIONS

This application claims priority to and benefit of U.S. Provisional Patent Application No. 63/353,751, filed Jun. 20, 2022, which is hereby incorporated by reference in its entirety.

### FIELD

The present disclosure generally relates to naproxen sodium tablets, and more particularly, to naproxen sodium tablets produced using a continuous process.

### BACKGROUND

Naproxen sodium is a non-steroidal anti-inflammatory drug (NSAID) used to treat inflammation associated with a variety of conditions as well as to provide long-lasting relief from mild to moderate pain. Typically, naproxen sodium is administered in a tablet drug form.

Conventional tableting processes require a granulation step when powders with poor flow properties are used. Use of low-flow powders can cause non-uniformity and segregation within product blends. Granulation is used to improve flowability of powdered ingredients by increasing particle size. Granulation can be accomplished using wet granulation, in which liquid binders are added to the powder, or dry granulation, in which powders are compressed or compacted and milled to achieve the desired properties. One commonly used dry granulation process is roller compaction. In roller compaction, a powdered blend is passed through two counter-rotating rollers that compact the powder into a ribbon, which is then milled into the desired granule size. Alternatively or in addition to granulation, glidants, such as colloidal silicon dioxide, may be added to a blend to enhance flow.

### SUMMARY

Described herein are naproxen sodium tablets without a glidant, and naproxen sodium tablets without a glidant produced using a continuous manufacturing process. Specifically, the continuous manufacturing process allows low flow materials, such as naproxen sodium, to flow freely without the addition of a glidant, such as colloidal silicon dioxide. As described above, conventional tableting processes require the use of a granulation process to transform a low-flow powder to granules that can more easily flow through the downstream process. However, by using the continuous tableting process described herein, which includes a loss-in-weight feeder particularly designed for low-flow materials, naproxen sodium tablets can be manufactured without the addition of a glidant. Further, this continuous process completely eliminates the need for a granulation process (e.g., wet granulation). Eliminating this pre-processing step can improve the cost and efficiency of producing naproxen sodium tablets.

Further, the continuous tableting process described herein includes a tablet press with three rollers facilitating direct compression. Traditional tablet presses contain two compression/compaction stations (pre-compression and main compression). Pre-compression rollers remove the air from the blend by compacting the blend providing densification, and the main compression rollers apply the force to form the tablets. Use of a third roller in this case being a second pre-compression roller, enhances the densification of the blend to an extent it compensates for the need of a pre-roller compacted granules. In other words, the third roller acts as a roller compactor in situ following pre and main compression of the blend to make the tablets.

In some embodiments, sodium naproxen tablets described herein can comprise excipients including stearic acid, sodium starch glycolate, mannitol, microcrystalline cellulose, magnesium stearate, or povidone. However, the sodium naproxen tablets described herein do not include a glidant (e.g., colloidal silicon dioxide).

In some embodiments, provided herein is an oral tablet comprising: naproxen sodium; mannitol; a superdisintegrant; and a lubricant, wherein the oral tablet does not comprise a glidant.

In some embodiments of the oral tablet, the superdisintegrant comprises sodium starch glycolate.

In some embodiments of the oral tablet, the oral tablet comprise 70-80 wt. % naproxen sodium.

In some embodiments of the oral tablet, the oral tablet comprises 15-25 wt. % mannitol.

In some embodiments of the oral tablet, the oral tablet comprises 3-8 wt. % superdisintegrant.

In some embodiments of the oral tablet, the oral tablet comprises 1-6 wt. % lubricant.

In some embodiments of the oral tablet, the lubricant comprises one or more of magnesium stearate or stearic acid.

In some embodiments of the oral tablet, the mannitol is spray-dried mannitol.

In some embodiments of the oral tablet, the oral tablet comprises 220 mg naproxen sodium.

In some embodiments, provided is a naproxen sodium tablet formed by the process of: mixing naproxen sodium, mannitol, a superdisintegrant, and a lubricant in a continuous in-line mixer to form a tableting mixture; transferring the tableting mixture to a tablet press; and pressing the tableting mixture into naproxen sodium tablets.

In some embodiments of the naproxen sodium tablet, the naproxen sodium tablet does not include a granulation step.

In some embodiments of the naproxen sodium tablet, mixing naproxen sodium, mannitol, a superdisintegrant, and lubricant in a continuous in-line mixer comprises feeding the naproxen sodium, the mannitol, the superdisintegrant, and the lubricant to the in-line mixer through a loss-in-weight feeder.

In some embodiments of the naproxen sodium tablet, mixing naproxen sodium, mannitol, a superdisintegrant, and lubricant in a continuous in-line mixer comprises mixing the naproxen sodium, the mannitol, and the superdisintegrant prior to adding the lubricant to the in-line mixer.

In some embodiments of the naproxen sodium tablet, transferring the tableting mixture to a tablet press comprises vacuuming transferring.

In some embodiments of the naproxen sodium tablet, the superdisintegrant comprises sodium starch glycolate.

In some embodiments of the naproxen sodium tablet, the lubricant comprises one or more of magnesium stearate or stearic acid.

In some embodiments of the naproxen sodium tablet, the oral tablet comprise 70-80 wt. % naproxen sodium.

In some embodiments of the naproxen sodium tablet, the oral tablet comprises 15-25 wt. % mannitol.

In some embodiments of the naproxen sodium tablet, the oral tablet comprises 3-8 wt. % superdisintegrant.

In some embodiments of the naproxen sodium tablet, the oral tablet comprises 1-6 wt. % lubricant.

In some embodiments of the naproxen sodium tablet, the mannitol is spray-dried mannitol.

In some embodiments of the naproxen sodium tablet, the oral tablet comprises 220 mg naproxen sodium.

In some embodiments, any one or more of the features, characteristics, or elements discussed above with respect to any of the embodiments may be incorporated into any of the other embodiments mentioned above or described elsewhere herein.

### BRIEF DESCRIPTION OF THE FIGURES

FIG. 1 shows a process for manufacturing naproxen sodium tablets, according to some embodiments;

FIG. 2 shows a loss-in-weight feeder designed for processing low-flow materials, according to some embodiments;

FIG. 3 shows volumetric decay with a twin screw at 80% screw speed across various agitator speeds based on a feeding trial for a continuous manufacturing process, according to some embodiments;

FIG. 4 shows volumetric decay with a twin-concave screw at 80% screw speed and 50% agitator speed based on a feeding trial for a continuous manufacturing process, according to some embodiments;

FIG. 5 shows gravimetric feeding data for a twin screw feeder based on a feeding trial for a continuous manufacturing process, according to some embodiments;

FIG. 6 shows gravimetric feeding data for a twin screw feeder based on a feeding trial for a continuous manufacturing process, according to some embodiments;

FIG. 7 shows gravimetric feeding data for a twin screw feeder based on a feeding trial for a continuous manufacturing process, according to some embodiments;

FIG. 8A shows an image of a hopper after running for two days, according to some embodiments; and

FIG. 8B shows an image of an entrance location of a hopper after running for two days, according to some embodiments.

### DETAILED DESCRIPTION

Described here are naproxen sodium tablets that do not include a glidant, and naproxen sodium tablets that do not include a glidant that are produced using a continuous manufacturing process. Specifically, the continuous manufacturing process is a tableting process that eliminates the need for a pre-processing granulation step, which is generally used to form tablets, particularly when working with low-flow materials such as naproxen sodium. Common granulation methods include roller compaction, for example. However, by manufacturing naproxen sodium tablets using the continuous tableting process described herein, naproxen sodium tablets may be produced in a more efficient and cost-effective manner. Specifically, the elimination of the granulation step saves a lot of time, waste, and equipment and maintenance costs. Further, the elimination of a glidant (e.g., colloidal silicon dioxide) saves the cost of that material. Fewer components in the final product also streamlines the manufacturing process and minimizes potential for error.

As mentioned above, naproxen sodium is a cohesive material with very poor flow properties. Due to the fineness and cohesive nature of naproxen sodium, it has been extremely challenging and unsuccessful in forming a free-flowing blend. Thus, naproxen sodium has conventionally been granulated using a wet or dry granulation process (roller compaction) before it is compressed into a tablet. Without the granulated naproxen sodium, blends of powdered naproxen sodium have very poor flow, which makes it impossible to make tablets (due to the blend sticking to punches) on a rotary tablet press.

Using the continuous manufacturing process described herein, poorly flowing materials such as naproxen sodium can be fed constantly and consistently by adopting various configurations of a loss in weight (LIW) feeder to achieve a free flowing, uniform blend using an in-line mixer that keeps the material/blend in a constant fluid motion by preventing any segregation and densification of the materials. The blend transported to the tablet press by a bulk transfer mechanism further ensures blend uniformity before it is compressed into tablets. The blend is also lubricated by magnesium stearate in the in-line mixer or by applying directly on to the punch tips through a controlled spraying mechanism allowing the blend to be compressed into tablets free of any physical defects.

### Naproxen Sodium Tablet Composition

### Active Pharmaceutical Ingredient: Naproxen Sodium

The tablets described herein include naproxen sodium (C₁₄H₁₄O₃), which is a non-steroidal anti-inflammatory and pain relieving pharmaceutical drug. Naproxen sodium can be difficult to process due to its low-flowing characteristics. In some embodiments, the naproxen sodium tablets described herein can include 50-90, 60-85, or 70-80 wt. % naproxen sodium. In some embodiments, the naproxen sodium tablets described herein can include less than or equal to 90, 85, 80, 75, 70, 65, 60, or 55 wt. % naproxen sodium. In some embodiments, the naproxen sodium tablets described herein can include more than or equal to 50, 55, 60, 65, 70, 75, 80, or 85 wt. % naproxen sodium.

In some embodiments, the naproxen sodium tablets comprising naproxen sodium may comprise one or more additional active pharmaceutical ingredients. For example, the oral tablets may comprise a single layer containing naproxen sodium, and one or more additional active pharmaceutical ingredients. Suitable additional active ingredients may include, but are not limited to, active pharmaceutical ingredients indicated for the treatment of pain, fever, and/or cold and flu, such as acetaminophen, phenylephrine, pseudoephedrine, doxylamine, dextromethorphan, and/or guaifenesin, or any pharmaceutically acceptable salt thereof (e.g., pseudoephedrine hydrochloride or pseudoephedrine sulfate).

### Excipients

As described herein, the oral tablets prepared using a continuous process may comprise the active pharmaceutical ingredient(s) (API) along with a plurality of excipients. Suitable excipients may include excipients such as binders, lubricants, distintegrants, and superdisintegrants. In some embodiments, the naproxen sodium tablets of the present disclosure produced using a continuous process further comprise excipients.

In some embodiments, the naproxen sodium tablet comprises mannitol as an excipient. Mannitol is a sugar alcohol that may serve as a diluent, a bulking agent, and/or a disintegrant. In some embodiments, the naproxen sodium tablet comprises 5-40, 10-30, or 15-25 wt. % mannitol. In some embodiments, the naproxen sodium tablet comprises less than or equal to 40, 35, 30, 25, 20, 15, or 10 wt. % mannitol. In some embodiments, the naproxen sodium tablet comprises more than or equal to 5, 10, 15, 20, 25, 30, or 35 wt. % mannitol. Suitable commercially-available mannitol can include Pearlitol SD200 by Roquette Freres.

In some embodiments, the certain properties or types of mannitol may be particularly useful. In some embodiments, the mannitol is spray-dried mannitol. In some embodiments of the foregoing, the mannitol has an average particle size of at least 50 μm, at least 75 μm, at least 100 μm, at least 125 μm or at least 150 μm. In other embodiments, the mannitol has an average particle size of less than or equal to 300 μm, less than or equal to 275 μm, less than or equal to 250 μm, less than or equal to 225 μm, or less than or equal to 200 μm. In certain embodiments, the mannitol has an average particle size of between 50 μm and 300 μm, between 50 μm and 250 μm, between 50 μm and 200 μm, between 100 μm and 300 μm, between 100 μm and 250 μm, between 100 μm and 200 μm, between 150 μm and 300 μm, between 150 μm and 250 μm, or between 50 μm and 200 μm.

In some embodiments, the naproxen sodium tablet comprises one or more superdisintegrants. For example, the naproxen sodium tablet may comprise sodium starch glycolate as an excipient. Sodium starch glycolate may be utilized as a superdisintegrant in pharmaceutical formulations to facilitate the conduction and penetration of dissolution media throughout the naproxen sodium tablet. In some embodiments, the naproxen sodium tablet comprises 0.1-20, 1-15, 1-10, or 3-8 wt. % superdisintegrants. In some embodiments, the naproxen sodium tablet comprises less than or equal to 20, 18, 16, 15, 14, 12, 10, 8, 6, 5, 4, 3, 2, or 1 wt. % superdisintegrant. In some embodiments, the naproxen sodium tablet comprises more than or equal to 0.1, 1, 2, 3, 4, 5, 6, 8, 10, 12, 14, 15, 16, or 18 superdisintegrant. Suitable commercially-available superdintegrants include that which is provided by DFE pharma.

In some embodiments, a naproxen sodium tablet comprises one or more lubricants. In some embodiments, the naproxen sodium tablet comprises magnesium stearate as an excipient. Magnesium stearate is an excipient used in various dosage forms as a lubricant to reduce sticking of powder material to processing equipment and facilitate the discharge of tablets from tablet presses. In some embodiments, the naproxen sodium tablet comprises 0.1-10, 1-5, or 1-2 wt. % lubricant. In some embodiments, the naproxen sodium tablet comprises less than or equal to 10, 9, 8, 7, 6, 5, 4, 3, 2, or 1 wt. % lubricant. In some embodiments, the naproxen sodium tablet comprises more than or equal to 0.1, 1, 2, 3, 4, 5, 6, 7, 8, or 9 wt. % lubricant. Suitable commercially-available lubricants can include that which is provided by Peter Greven Fett-Chemie.

In some embodiments, a naproxen sodium tablet comprises stearic acid. Like magnesium stearate, stearic acid acts as a lubricant. Specifically, stearic acid is both a lubricant and a solubilizing agent, which can help to achieve the desired flowability of powder mixtures during manufacture as well as dissolution profile in actual usage of the tablet. In some embodiments, the naproxen sodium tablets comprise 0.1-10, 1-5, or 2-4 wt. % stearic acid. In some embodiments, the naproxen sodium tablets comprise less than or equal to 10, 9, 8, 7, 6, 5, 4, 3, 2, or 1 wt. % naproxen sodium. In some embodiments, the naproxen sodium tablets comprise greater than or equal to 0.1, 1, 2, 3, 4, 5, 6, 7, 8, or 9 wt. % stearic acid.

In some embodiments, a naproxen sodium tablet as described herein can include 70-75 wt. % naproxen sodium, 15-25 wt. % mannitol, 3-8 wt. % superdisintegrant such as sodium starch glycolate, and 1-2 wt. % magnesium stearate.

In some embodiments, a naproxen sodium tablet as described herein can include 73-74 wt. % naproxen sodium, 19-21 wt. % mannitol, 4-6 wt. % superdisintegrant such as sodium starch glycolate, and 1-2 wt. % magnesium stearate.

In some embodiments, a naproxen sodium tablet as described herein can include 73.33 wt. % naproxen sodium, 20 wt. % mannitol, 5 wt. % superdisintegrant such as sodium starch glycolate, and 1.67 wt. % magnesium stearate.

Two examples of a naproxen sodium formulation according to some embodiments are shown in the tables below.

| | mg/Tab | % |
|---|---|---|
| Naproxen Sodium, USP | 220 | 73.33 |
| Mannitol | 60 | 20.00 |
| Sodium Starch Glycolate | 15 | 5.00 |
| Magnesium Stearate | 5 | 1.67 |
| Total: | 300 | 100 |

| | mg/Tab | % |
|---|---|---|
| Naproxen Sodium, USP | 220 | 72.6 |
| Mannitol | 60 | 19.8 |
| Sodium Starch Glycolate | 15 | 4.95 |
| Magnesium Stearate | 5 | 1.65 |
| Stearic Acid | 3 | 0.99 |
| Total: | 303 | 100 |

### Naproxen Sodium Tablet Specifications

Described below are several target specifications for naproxen sodium tablets manufactured using a continuous manufacturing process, according to some embodiments. Specifically, the target specifications described include target tablet weight, individual tablet weight range, average tablet weight range, tablet thickness, tablet diameter, tablet hardness, and naproxen sodium content.

Tablet Core properties ALEVE (Naproxen Sodium Tablets)

| Target tablet weight | 300 | mg |
|---|---|---|
| Individual tablet weight range | (+/−5%) (15 mg) | |
| Average tablet weight range (10 tbas) | (+/−7%) (21 mg) | |
| Tablet thickness | 4.68-5.38 | mm |
| Tablet diameter | 10 | mm |

| | |
|---|---|
| Tablet hardness | 80-120N |
| Naproxen sodium content | 90-110% |

### Methods of Manufacturing Naproxen Sodium Tablets

The naproxen sodium tablets provided herein do not include a glidant such as colloidal silicon dioxide. In conventional tableting processes, a granulation step is required, particularly for low-flowing materials such as naproxen sodium. A glidant can be introduced during the granulation step to improve the flowability of the material and thus, increase the processability of the material downstream. Here, the naproxen sodium tablets are manufactured using a continuous manufacturing process that explicitly eliminates a pre-processing granulation step, and thus, also eliminates the addition of a glidant. Accordingly, the sodium naproxen tablets described herein do not include a glidant in their formulation.

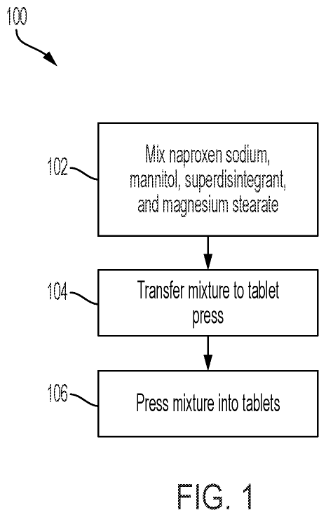

FIG. 1 shows an example of a process 100 for manufacturing naproxen sodium tablets, according to some embodiments described herein. The process can include a loss-in-weight (LIW) feeder, an in-line (continuous) mixer, and a tablet press.

At step 102, the naproxen sodium, mannitol, superdisintegrant (e.g., sodium starch glycolate), and magnesium stearate are mixed. In some embodiments, these components are all introduced into the in-line mixer at the same time. In some embodiments, these components are introduced into the mixer at different times.

For example, the naproxen sodium, mannitol, and superdisintegrant may be fed into the in-line mixer via the LIW feeder first and mixed. A lubricant, such as magnesium stearate, may then be added to the in-line mixer via the LIW feeder and mixed with the naproxen sodium-mannitol-superdisintegrant mixture.

In some embodiments, the naproxen sodium and superdisintegrant are mixed first, and the mannitol is added to the in-line mixer at a later time. Magnesium stearate is added as an independent component. In some embodiments, the naproxen sodium and mannitol mixed first, and the superdisintegrant is added to the in-line mixer at a later time. Magnesium stearate is added as an independent component. In some embodiments, the mannitol and superdisintegrant are mixed first, and the naproxen sodium and is added to the in-line mixer at a later time. Magnesium stearate is added as an independent component.

By using a LIW feeder specifically designed for low-flow materials, the components can be added accurately and consistently to the in-line mixer, irrespective of their cohesivity properties, and without the addition of a glidant. Additionally, the use of an in-line continuous mixer allows the material to maintain a constant fluidized state and a consistent flow during the manufacturing process to prevent segregation and densification of the blend.

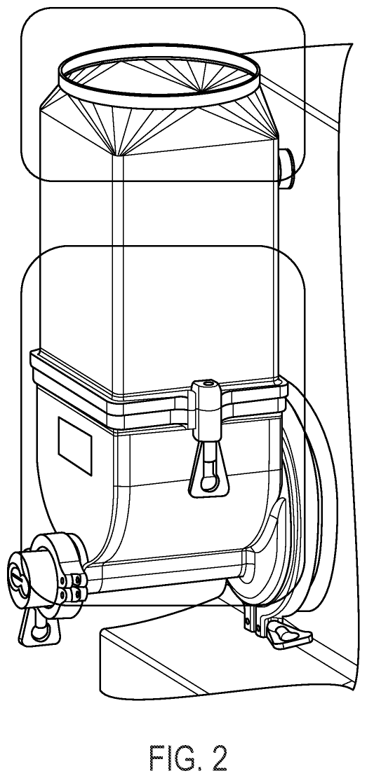

FIG. 2 shows one embodiment of a LIW feeder 200 that may be used in a continuous process for manufacturing naproxen sodium tablets without the addition of a glidant, as described herein. Loss-in-weight feeders are gravimetric feeders that measure a material's weight to achieve and maintain a predetermined feed rate.

The LIW feeder depicted in FIG. 2 is specifically designed for processing low-flow materials.

At step 104, the naproxen sodium-mannitol-superdisintegrant-magnesium stearate mixture (i.e., the tableting mixture) is transferred to a tablet press using a bulk transferring mechanism. A bulk transferring mechanism allows the blend to remain uniform for tablet pressing. In some embodiments, the tableting mixture is transferred to a tablet press using a vacuum transferring technique.

At step 106, the tableting mixture is pressed into naproxen sodium tablets using a tablet press. In some embodiments, the tablet press may be a FE55 tablet press by Fette Compacting.

In some embodiments, using the continuous manufacturing process described herein, 100-2000, 100-1000, 100-500, 200-400, or 300-350 tablets can be produced per hour. In some embodiments, the continuous manufacturing process described herein can yield less than or equal to 2000, 1500, 1000, 900, 800, 700, 600, 500, 400, 350, 300, 250, 200, or 150 tablets per hour. In some embodiments, the continuous manufacturing process described herein can yield more than or equal to 100, 150, 200, 250, 300, 350, 400, 500, 600, 700, 800, 900, 100, or 1500 tablets per hour. In some embodiments, the continuous manufacturing process described herein can yield 320 tablets/hour.

### Example: Manufacturing a Naproxen Sodium Tablet Using a Continuous Manufacturing Process

220 mg naproxen sodium, 60 mg mannitol, and 15 mg sodium starch glycolate were fed into an in-line continuous mixer from a LIW feeder and mixed. 5 mg magnesium stearate was added to the in-line mixer from the LIW feeder and the naproxen sodium, mannitol, and sodium starch glycolate mixture was further mixed with the magnesium stearate to form a tableting mixture.

The tableting mixture was vacuum-transferred to a FE55 tablet press by Fette Compacting with 10 mm round standard concave punches.

The tableting mixture was compressed into tablets on a high speed rotary tablet press (Fette's FE55) which includes three compression rollers. Standard tablet presses generally include only two compression rollers (pre-compression and main compression). The pre compression force by the pre-compression roller can reduce the entrapped air that can impact the tablet integrity. The main compression force by the main compression roller can impact the hardness of the tablet. The second pre-compression roller present on FE55 (i.e., the third roller) is used to control the de-aeration but also to adjust the tablet hardness in conjunction with the first pre-compression roller. This in turn helps the main compression roller to compress tablets to the required hardness.

### Lubricating Punch Tips of Tablet Press

As described above, in some embodiments, the lubricant (e.g., magnesium stearate) is added directly to the in-line mixer. However, in other embodiments, the lubricant may be applied directly to the punch tips of the tablet press to prevent sticking of the tableting mixture to the punch tips of the tablet press. In some embodiments, the lubricant can be applied to the punch tips using a controlled spraying technique. External lubrication refers to the application of lubricant to the tablet surface only through a device with compressed air and a powder delivery point to the punch tips. In some embodiments, some lubricant is added to the in-line mixer and some lubricant is added to the punch tips of the tablet press.

### EXAMPLES

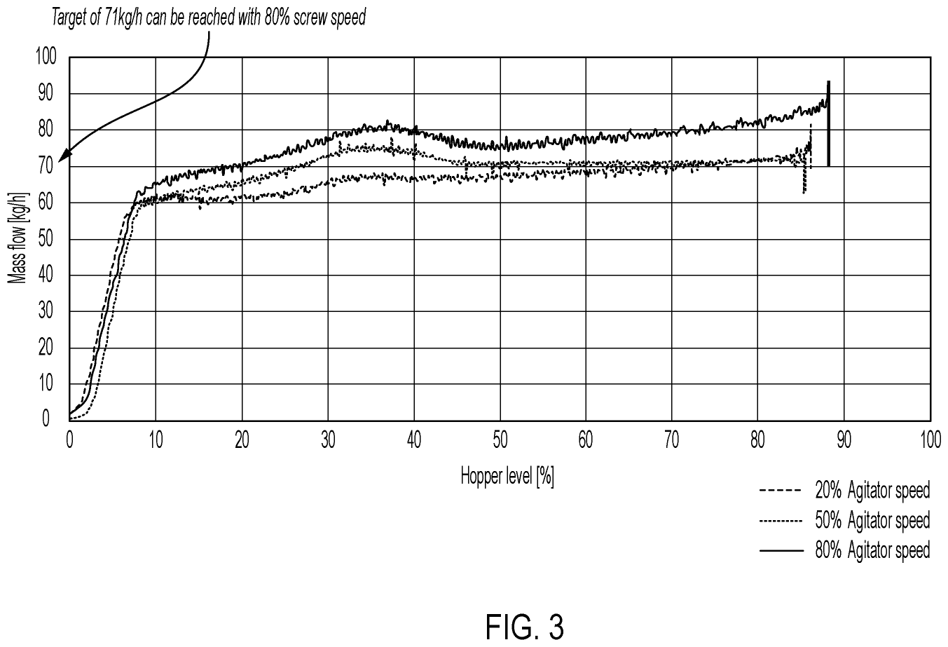

The graphs shown in FIGS. 3-7 show naproxen sodium feeding data under various conditions to achieve a mass flow rate of 71.094 kg/hr (volume flow of 151.26 dm³/h) in order to produce naproxen sodium tablets to a target weight of 300 mg at a production speed of 320,000 tablets/hr (98 kg/hr). All trials were executed using only naproxen sodium. The performance of LIW feeder is dependent on the density of the materials and density of naproxen sodium was determined to be 0.47 kg/dm³ (unless otherwise indicated). Naproxen sodium has medium to poor flowability characteristics, fluidizes easily, and de-aerates quickly. A TS2024 twin-spiral screw was used as indicated, and a TC2020 twin-concave screw was used as indicated. The feeder used was a DDSR20-QR (6 kg load cell, 7 Liter hopper volume).

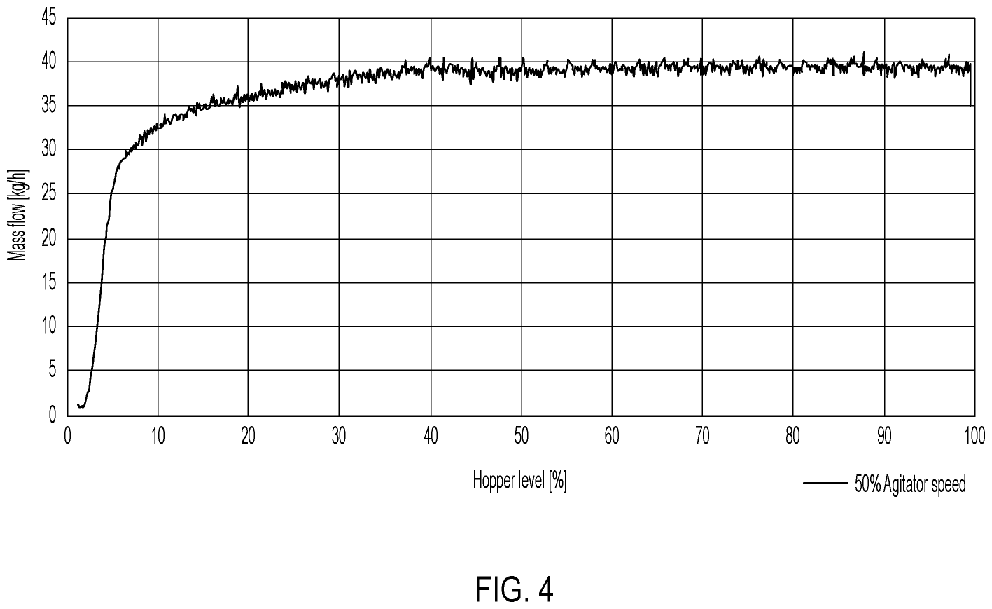

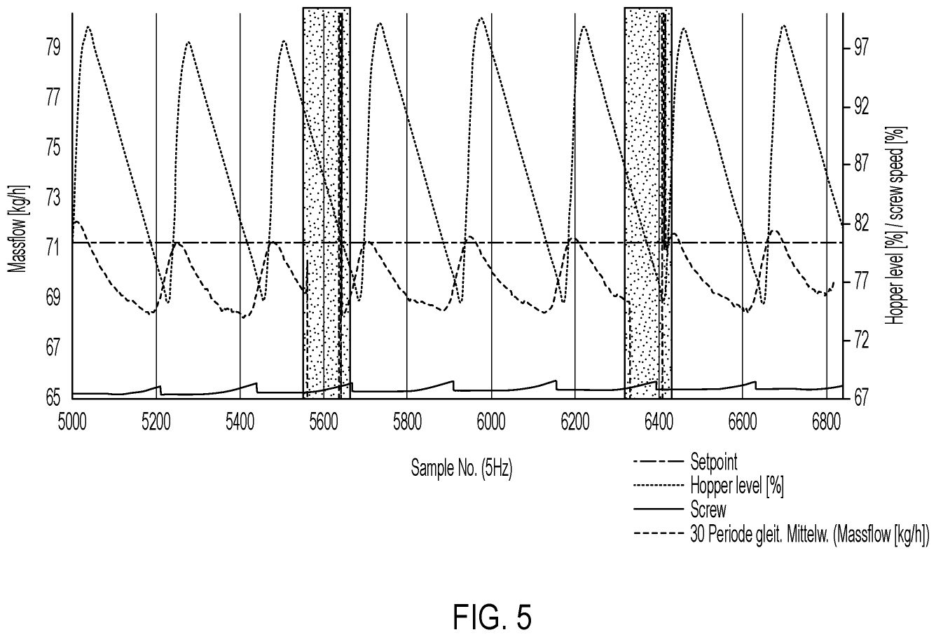

FIGS. 3 and 4 show volumetric decay data. This type of test includes checking the maximum feed rate, analyzing the impact of hopper fill level on screw filling, and determining the optimal refill levels. FIGS. 5-7 show gravimetric data, which includes analyzing the impact of refilling and checking the overall gravimetric feed accuracy.

FIG. 3 shows volumetric decay with a TS2024 twin screw at 80% screw speed across various agitator speeds (20%, 50%, and 80%), according to some embodiments. As shown in the graph, a target of 71 kg/h can be achieved at 80% screw speed. The behavior depicted at 30-40% hopper level can be attributed to the influence of the agitator, when the powder surface starts to move inside the hopper. This is a unique behavior of naproxen sodium.

FIG. 4 shows volumetric decay with a TS2020 twin-concave screw at 80% screw speed and 50% agitator speed, according to some embodiments. As shown in the graph, the twin-concave screw does not show the nuanced behavior of naproxen sodium depicted at 30-40% hopper level of FIG. 3. Further, a target of 35 kg/h can be achieved with a single twin-concave screw at 80% screw speed.

FIG. 5 shows gravimetric feeding data for a TS2024 twin screw, according to some embodiments. The mass flow rate of this trial was 71.094 kg/h. The two shaded bars running vertically through the graph shows when the bin was exchanged on the reference scale. The peaks in the mass flow (approximately a 4% overshoot) indicate a refill.

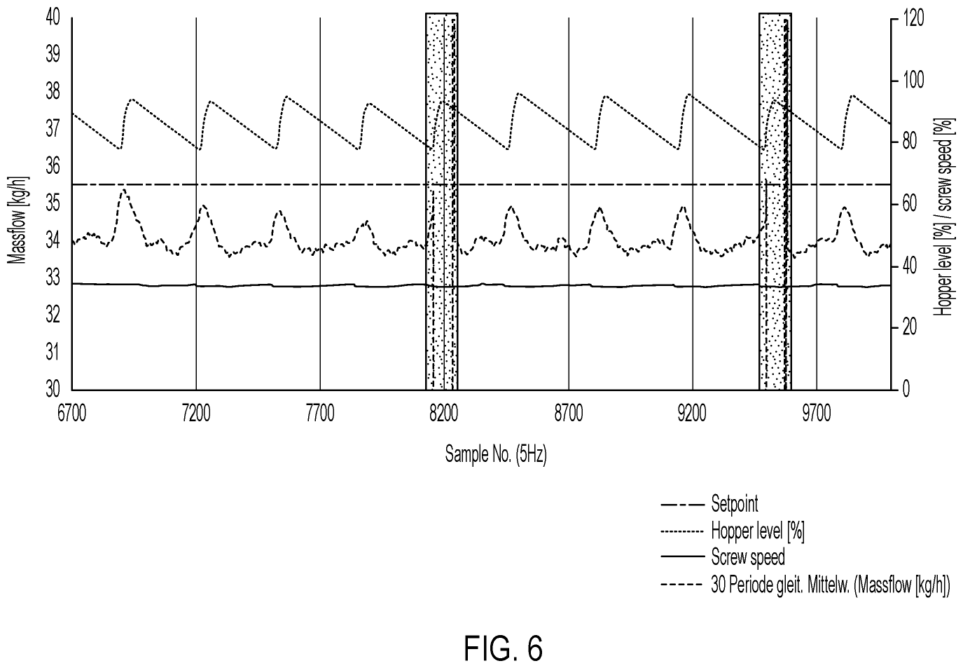

FIG. 6 shows gravimetric feeding data for a TS2024 twin screw, according to some embodiments. The mass flow rate of this trial was 35.547 kg/h. The two shaded bars running vertically through the graph shows when the bin was exchanged on the reference scale. The peaks in the mass flow (approximately a 2.5% overshoot) indicate a refill.

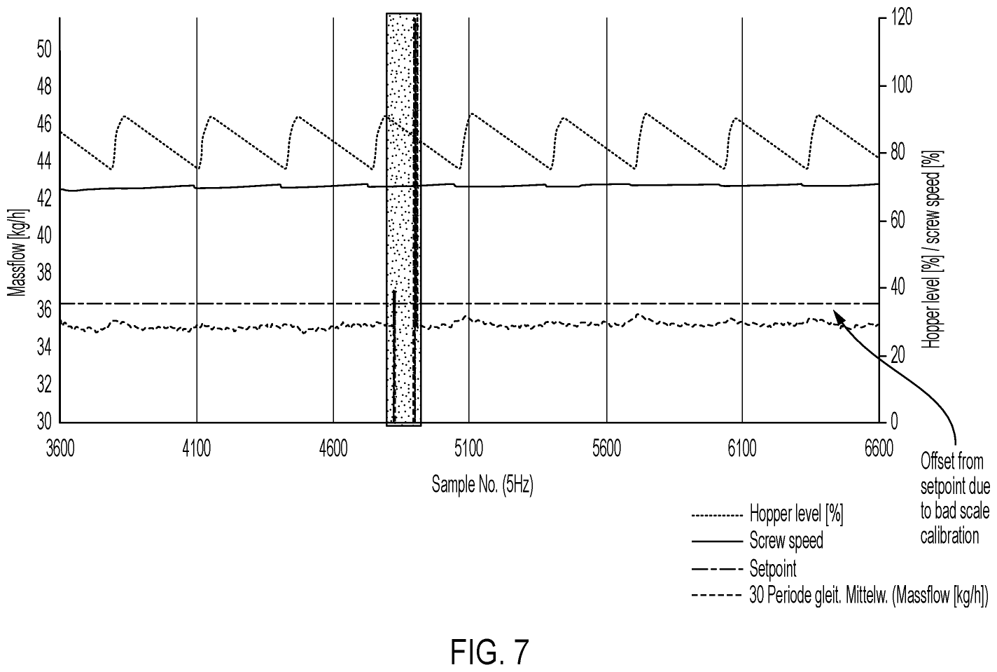

FIG. 7 shows gravimetric feeding data for a TC2020 twin-concave screw, according to some embodiments. The shaded bar running vertically through the graph shows when the bin was exchanged on the reference scale. As shown, there is almost no impact of refilling shown in the mass flow data line.

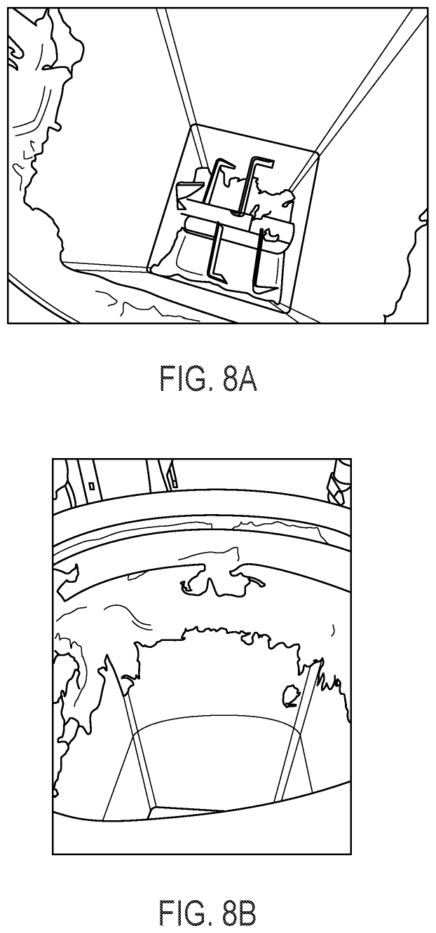

Both of the images depicted in FIGS. 8A and 8B were taken immediately after running empty. Specifically, FIG. 8A shows an image of a LIW feeder after running for two days, according to some embodiments. As shown, there is little naproxen sodium power remaining in the hopper. Thus, this shows that there is no sticking of the naproxen sodium powder to the walls after running two days without cleaning. The hopper also remained static overnight filled with powder, and it still did not stick. FIG. 8B shows an image of an entrance location of a hopper after running for two days, according to some embodiments. As shown there is some powder/dust accumulation, but very little, along the upper corners of the hopper after

The foregoing description sets forth exemplary systems, methods, techniques, parameters, and the like. It should be recognized, however, that such description is not intended as a limitation on the scope of the present disclosure but is instead provided as a description of exemplary embodiments.

Although the description herein uses terms first, second, etc. to describe various elements, these elements should not be limited by the terms. These terms are only used to distinguish one element from another.

1. An oral tablet comprising: naproxen sodium; mannitol; a superdisintegrant; and a lubricant, wherein the oral tablet does not comprise a glidant.

2. The oral tablet of claim 1, wherein the superdisintegrant comprises sodium starch glycolate.

3. The oral tablet of claim 1, wherein the oral tablet comprises 70-80 wt. % naproxen sodium.

4. The oral tablet of claim 1, wherein the oral tablet comprises 15-25 wt. % mannitol.

5. The oral tablet of claim 1, wherein the oral tablet comprises 3-8 wt. % superdisintegrant.

6. The oral tablet of claim 1, wherein the oral tablet comprises 1-6 wt. % lubricant.

7. The oral tablet of claim 1, wherein the lubricant comprises one or more of magnesium stearate or stearic acid.

8. The oral tablet of claim 1, wherein the mannitol is spray-dried mannitol.

9. The oral tablet of claim 1, wherein the oral tablet comprises 220 mg naproxen sodium.

10. A naproxen sodium tablet formed by the process of:
mixing naproxen sodium, mannitol, a superdisintegrant, and a lubricant in a continuous in-line mixer to form a tableting mixture;
transferring the tableting mixture to a tablet press; and
pressing the tableting mixture into naproxen sodium tablets.

11. The naproxen sodium tablet of claim 10, wherein the process does not include a granulation step.

12. The naproxen sodium tablet of claim 10, wherein mixing naproxen sodium, mannitol, a superdisintegrant, and lubricant in a continuous in-line mixer comprises feeding the naproxen sodium, the mannitol, the superdisintegrant, and the lubricant to the in-line mixer through a loss-in-weight feeder.

13. The naproxen sodium tablet of claim 10, wherein mixing naproxen sodium, mannitol, a superdisintegrant, and lubricant in a continuous in-line mixer comprises mixing the naproxen sodium, the mannitol, and the superdisintegrant prior to adding the lubricant to the in-line mixer.

14. The naproxen sodium tablet of claim 10, wherein transferring the tableting mixture to a tablet press comprises vacuuming transferring.

15. The naproxen sodium tablet of claim 10, wherein the superdisintegrant comprises sodium starch glycolate.

16. The naproxen sodium tablet of claim 10, wherein the lubricant comprises one or more of magnesium stearate or stearic acid.

17. The naproxen sodium tablet of claim 10, wherein the oral tablet comprise comprises 70-80 wt. % naproxen sodium.

18. The naproxen sodium tablet of claim 10, wherein the oral tablet comprises 15-25 wt. % mannitol.

19. The naproxen sodium tablet of claim 10, wherein the oral tablet comprises 3-8 wt. % superdisintegrant.

20. The naproxen sodium tablet of claim 10, wherein the oral tablet comprises 1-6 wt. % lubricant.

21. The naproxen sodium tablet of claim 10, wherein the mannitol is spray-dried mannitol.

22. The naproxen sodium tablet of claim 10, wherein the oral tablet comprises 220 mg naproxen sodium.
